(19) United States
(12) Patent Application Publication
(10) Pub. No.: US 2019/0307703 A1
(43) Pub. Date: Oct. 10, 2019

(54) MULTI-FUNCTIONAL NANOPARTICLES FOR VACCINATION

(71) Applicant: Yuan Zhang, Saunderstown, RI (US)

(72) Inventor: Yuan Zhang, Saunderstown, RI (US)

(21) Appl. No.: 16/230,148

(22) Filed: Dec. 21, 2018

Related U.S. Application Data
(63) Continuation-in-part of application No. 15/979,874, filed on May 15, 2018.
(60) Provisional application No. 62/655,012, filed on Apr. 9, 2018.

(51) Int. Cl. A61K 9/51; A61K 39/12; A61K 39/39; A61K 39/00

(52) U.S. Cl. CPC A61K 9/51; A61K 39/12; A61K 39/39; A61K 2039/51; A61K 2039/55555; A61K 2039/55516; A61K 39/0012

(57) ABSTRACT

The present invention generally relates to a dynamic nanoparticle used for vaccination. Specifically, the claimed product comprises of an adaptive nanoparticle wherein both the outer surface and the inner core are customizable for targeted application.

### FIELD OF INVENTION

The present invention generally relates to a dynamic nanoparticle used for vaccination. Specifically, the claimed product comprises of an adaptive nanoparticle wherein both the outer surface and the inner core are customizable for targeted application.

### BACKGROUND OF THE INVENTION

After the successful eradication of small pox, vaccine development followed the “isolate, inactivate, inject” paradigm developed by Louis Pasteur. According to this model, a weakened or attenuated form of an infectious agent is injected into a subject in order to develop a natural immunity against the more virulent form of the infectious agent. However, despite the triumphs achieved by this approach, the safety risk associated with using a live infectious agent, irrespective of how weakened it may be, remains. Furthermore, there are many more infectious diseases that continue to thrive as un-preventable under the live vaccine scheme. For example, infections that lead to viral latency (i.e. HIV-1, hepatitis C virus) or those infections that have a high degree of variability (i.e. dengue, HIV-1) have been unaffected by vaccine development according to the Pasteur model. Accordingly, what is needed are viable vaccines that protect by mechanisms beyond inducing natural immunity yet are highly efficient and safe.

To address the above limitations, vaccines have evolved to rely upon isolated proteins, polysaccharides, or naked DNA in encoding protective antigens. While this approach has led to less reactogenic—and thereby safer—vaccines, overall the resulting vaccines are poor immunogens. Accordingly, such vaccines require adjuvants to boost their efficacy. However, this raises another problem—adjuvants, especially aluminum based—can induce local reactions and fail to generate strong cell-mediated immunity.

Thus, to maintain safety but elevate efficacy, nanoparticles have been engineered as delivery vehicles for various therapeutic cargos including nucleic acids. Typically 25 to 200 nm in diameter and varying in composition, size, shape, and surface proteins, nanoparticle vaccines are proving to be effective in both prophylactic and therapeutic approaches. In some cases, the nanoparticle vaccine functions by using the nanoparticle as the vehicle for antigens. In the case of antigen-loaded nanoparticle vaccines, the vaccine enables site directed delivery, the prolonged release of the antigens, and protects the antigens against cellular degradation before inducing an immune response. Furthermore, enclosing the antigen within a nanoparticle replicates the natural presentation of the antigen as presented by a pathogen since the nanoparticles are structurally similar to viruses. Nanoparticles may also promote cross-presentation by following the endocytic pathway through endosome disruption to release loaded antigens, adjuvants, or both, to the cytosol, where they can be processed and presented intracellularly to trigger cytotoxic T lymphocyte (CTL) killing effects.

Currently there are numerous nanoparticle-based vaccines that have been examined for utility. Many such vaccines have demonstrated encouraging efficacy against infectious agents and maladies such as malaria, influenza, Ebola, and HIV. Despite the advances that have been made, there is a need in the arts for improving the design of nanoparticle vaccines such that the nanoparticle-based virus mimicry can enhance an immune response.

### SUMMARY OF THE INVENTION

In general, nanoparticle transfection is preferred over other transfection methods due to its sustained release characteristics and enhanced safety profile and biocompatibility over virus-mediated gene transfer. Nanoparticles efficiently enter cells by exploiting the endocytosis pathway, followed by the entrapment in the acidic endosomal and lysosomal compartments for degradation. To prevent enzymatic degradation intracellularly, certain endosome-disrupting agents or rationally designed biomaterials (i.e. lipids, polymers) are usually incorporated into the nanoparticle carriers in order to promote the release of therapeutic cargos (i.e. nucleic acids) entrapped in nanoparticles from endosomes or lysosomes to cytoplasm in order to exert their biological functions.

Nanoparticles are small (generally under 200 nanometers) objects that are either naturally or artificially produced, and despite their small size, have a relatively large surface for cellular adhesion. Further, nanoparticles have an enhanced propensity for transport into a cell and have the ability to shield incorporated nucleic acid from degradation by nucleases.

In one respect, the present invention is directed to synthesized nanoparticles for use as carriers for nucleic acid, antigens, proteins and peptides. It is another aspect of the present invention to provide a synthesized nanoparticle having the ability to carry nucleic acid adjuvants within the nanoparticle while concurrently carrying protein or peptide antigens on the surface of the nanoparticle or embedded within the outer surface of the nanoparticle. These and other features and advantages of the present invention will be explained and will become obvious to one skilled in the art through the description of the invention that follows.

Alphavirus replicon RNA may serve as an adjuvant to promote the immunogenicity of engineered peptide subunit vaccines. RNA replicons contain the genes encoding alphavirus RNA replication machinery (non-structure proteins), but lack genes encoding the viral structural proteins required to make an infectious alphavirus particle. The structural protein genes are replaced with genes encoding antigens of interest for vaccine applications. Alphavirus replicon-based vectors, such as Sindbis virus (SIN), Semliki Forest virus (SFV) and Venezuelan equine encephalitis virus (VEE), can trigger the production of type I interferon (IFN) and stimulate innate signaling pathways. Naked replicon vectors are prone to enzymatic degradation by nucleases in vivo. While viral particle-based delivery systems may induce undesirable anti-vector immunity and raise significant safety issues due to the possibility of genome alteration, Replicon RNAs loaded in synthetic nanoparticles are expected to elicit increased antigen production and immunogenicity in vivo, without safety concerns. The integration of nanoparticle-formulated alphavirus replicon components into nanoparticle-based vaccines loaded with antigens and adjuvants, either separately or co-encapsulated, promotes immune responses and vaccine efficacy. In some instances, nanoparticle-based vaccines loaded solely with antigens promote immune responses and vaccine efficacy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Accompanying this written specification is a collection of drawings of exemplary embodiments of the present invention. One of ordinary skill in the art would appreciate that these are merely exemplary embodiments, and additional and alternative embodiments may exist and still be within the scope and spirit of the invention as described herein.

FIG. 1 shows a Transmission Electron Microscopy (TEM) image of LNP-adjuvants, where the scale bar is 100 nm.

FIG. 2A shows a graph comparing the ability of the LNP-adjuvant, specifically LNP-polyIC, against the ability of non-formulated polyIC to produce type I interferon (IFN) after an incubation period of twenty-four hours in vitro.

FIG. 2B shows a graph comparing the ability of the LNP-adjuvant, specifically LNP-CpG, against the ability of non-formulated CpG to produce type I interferon (IFN) after an incubation period of twenty-four hours in vitro.

FIG. 2C shows a graph comparing the ability of the LNP-adjuvant, specifically LNP-Replicon, against the ability of non-formulated Replicon to produce type I interferon (IFN) after an incubation period of twenty-four hours in vitro.

FIG. 2D shows a graph comparing the ability of the LNP-adjuvant, specifically LNP-CDN, against the ability of non-formulated CDN to produce type I interferon (IFN) after an incubation period of twenty-four hours in vitro.

FIG. 2E shows a graph demonstrating the optimal dosage of LNP-polyIC tested to induce the production of IFN after an incubation period of twenty-four hours in vitro.

FIG. 2F shows a graph demonstrating the optimal dosage of LNP-CpG tested to induce the production of IFN after an incubation period of twenty-four hours in vitro.

FIG. 3A shows a graph demonstrating that the tested LNP-adjuvants (LNP-polyIC, LNP-CDN, LNP-CpG, and LNP-Replicon) stimulate the activation of antigen-presenting cells (APCs), particularly cross-presenting dendritic cell (DC), in draining lymph nodes (dLNs) after immunization.

FIG. 3B shows a graph demonstrating that the tested LNP-adjuvants (LNP-polyIC, LNP-CDN, LNP-CpG, and LNP-Replicon) stimulate the activation of antigen-presenting cells (APCs), particularly macrophage, in draining lymph nodes (dLNs) after immunization.

FIG. 4A shows a graph demonstrating that the tested LNP-adjuvants (LNP-polyIC, LNP-CDN, LNP-CpG, and LNP-Replicon) promote antigen-specific T-cell response after immunization. Particularly, that LNP-adjuvants promote T-cell activation induced degranulation, as evidenced by the CD107 expression in CD8⁺ T cells in peripheral blood.

FIG. 4B shows a graph demonstrating that the tested LNP-adjuvants (LNP-polyIC, LNP-CDN, LNP-CpG, and LNP-Replicon) promote antigen-specific T-cell response after immunization. Particularly, that LNP-adjuvants promote T-cell cytolytic killing effect, as evidenced by the granzyme B expression in CD8⁺ T cells in peripheral blood.

FIG. 4C shows a graph demonstrating that the tested LNP-adjuvants (LNP-polyIC, LNP-CDN, LNP-CpG, and LNP-Replicon) promote antigen-specific T-cell response after immunization. Particularly, that LNP-adjuvants enhance T-cell effector function, as evidenced by the production of pro-inflammatory cytokine IFNγ in CD8⁺ T cells in peripheral blood.

FIG. 4D shows a graph demonstrating that the tested LNP-adjuvants (LNP-polyIC, LNP-CDN, LNP-CpG, and LNP-Replicon) promote antigen-specific T-cell response after immunization. Particularly, that LNP-adjuvants enhance T-cell effector function, as evidenced by the production of pro-inflammatory cytokine TNFα in CD8⁺ T cells in peripheral blood.

FIG. 4E shows a graph demonstrating that the tested LNP-adjuvants (LNP-polyIC, LNP-CDN, LNP-CpG, and LNP-Replicon) promote antigen-specific T-cell response after immunization. Particularly, that LNP-adjuvants enhance antigen-specific tetramer positive CD8⁺ T-cell clone in peripheral blood.

FIG. 5A shows a graph demonstrating that the disclosed LNP-Adjuvants inhibit tumor progression in melanoma-bearing mice when immunized with DSPE-PEG-antigen (Ag) EGP conjugate (amph-EGP).

FIG. 5B shows a graph demonstrating that the disclosed LNP-Adjuvants prolong the survival of melanoma-bearing mice when immunized with amph-EGP.

FIG. 6 shows a Transmission Electron Microscopy (TEM) image of LNP-(polyIC+(amph-Ag)), where the scale bar is 100 nm.

FIG. 7A is an illustration of separate antigen (Ag) and adjuvant components ((LNP-polyIC)+(amph-Ag))

FIG. 7B is an illustration of an antigen (Ag) and adjuvant combined nanoparticle (LNP-(polyIC+(amph-Ag)).

FIG. 7C is a graph comparing the expression of proliferation marker Ki67 in CD8⁺ T-cells from PBMC in untreated cells, in cells treated with unformulated adjuvant and antigen components (polyIC+(amph-Ag)), in cells treated with separate antigen and adjuvant components ((LNP-polyIC)+(amph-Ag)), and in cells treated with antigen and adjuvant combined nanoparticles (LNP-(polyIC+(amph-Ag)).

FIG. 7D is an illustrative graph comparing the expression of the degranulation marker CD107 in CD8⁺ T cells from PBMC in untreated cells, in cells treated with unformulated adjuvant and antigen components (polyIC+(amph-Ag)), in cells treated with separate antigen and adjuvant components ((LNP-polyIC)+(amph-Ag)), and in cells treated with antigen and adjuvant combined nanoparticles (LNP-(polyIC+(amph-Ag)).

FIG. 7E is a statistical plot comparing the expression of the degranulation marker CD107 in CD8⁺ T cells from PBMC in untreated cells, in cells treated with unformulated adjuvant and antigen components (polyIC+(amph-Ag)), in cells treated with separate antigen and adjuvant components ((LNP-polyIC)+(amph-Ag)), and in cells treated with antigen and adjuvant combined nanoparticles (LNP-(polyIC+(amph-Ag)).

FIG. 7F is an illustrative graph comparing the expression of cytolytic enzyme granzyme B in CD8⁺ T cells from PBMC in untreated cells, in cells treated with unformulated adjuvant and antigen components (polyIC+(amph-Ag)), in cells treated with separate antigen and adjuvant components ((LNP-polyIC)+(amph-Ag)), and in cells treated with antigen and adjuvant combined nanoparticles (LNP-(polyIC+(amph-Ag)).

FIG. 7G is a statistical plot comparing the expression of cytolytic enzyme granzyme B in CD8⁺ T cells from PBMC in untreated cells, in cells treated with unformulated adjuvant and antigen components (polyIC+(amph-Ag)), in cells treated with separate antigen and adjuvant components ((LNP-polyIC)+(amph-Ag)), and in cells treated with antigen and adjuvant combined nanoparticles (LNP-(polyIC+(amph-Ag)).

FIG. 7H is an illustrative graph of the production of pro-inflammatory cytokines IFNγ in CD8⁺ T cells from PBMC in untreated cells, in cells treated with unformulated adjuvant and antigen components (polyIC+(amph-Ag)), in cells treated with separate antigen and adjuvant components ((LNP-polyIC)+(amph-Ag)), and in cells treated with antigen and adjuvant combined nanoparticles (LNP-(polyIC+(amph-Ag)).

FIG. 7I is a statistical plot of the production of pro-inflammatory cytokines IFNγ in CD8⁺ T cells from PBMC untreated cells, in cells treated with unformulated adjuvant and antigen components (polyIC+(amph-Ag)), in cells treated with separate antigen and adjuvant components ((LNP-polyIC)+(amph-Ag)), and in cells treated with antigen and adjuvant combined nanoparticles (LNP-(polyIC+(amph-Ag)).

FIG. 7J is an illustrative graph of the production of pro-inflammatory cytokines TNFα in CD8⁺ T cells from PBMC.

FIG. 7K is a statistical plot of the production of pro-inflammatory cytokines TNFα in CD8⁺ T cells from PBMC.

### DETAILED DESCRIPTION OF THE INVENTION

In the Summary above, the Detailed Description, the claims below, and in the accompanying drawings, reference is made to particular features of the present invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Whenever a reference herein is made to a method comprising two or more defined steps, the defined steps can be carried out in any order or simultaneously (except where the context excludes that possibility), and the method can include one or more other steps which are carried out before any of the defined steps, between two of the defined steps, or after all of the defined steps (except where context excludes that possibility).

Abbreviations used throughout this description have the following meaning:
CDN: cyclic dinucleotides
CpG: unmethylated single stranded DNA
DAPI: 2-(4-amidinophenyl)-1H-indole-6-carboxamidine
DDAB: dimethyldioctadecylammonium
DGTS: 1,2-dipalmitoyl-sn-glycero-3-O-4 ′-(N,N,N-trimethyl)-homoserine
DGTS-d9: 1,2-dipalmitoyl-sn-glycero-3-O-4′-[N,N,N-trimethyl(d9)]-homoserine
DMG-PEG: 1,2-dimyristoyl-rac-glycero-3-methylpolyoxyethylene
DMPE-PEG: N-(methylpolyoxyethylene oxycarbonyl)-1,2-dimyristoyl-sn-glycero-3-phosphoethanolamine
DOG-PEG: 1,2-dioleoyl-rac-glycerol methoxypolyethylene glycol
DOPA: 1 2-dioleoyl-sn-glycero-3-phosphate
DOPC: 1,2-dioleoyl-sn-glycero-3-phosphocholine
DOPE: 1 2-dioleoyl-sn-glycero-3-phosphoethanolamine
DOTAP: 1,2-dioleoyl-3-trimethylammonium-propane
DOTMA: 1,2-di-O-octadecenyl-3-trimethylammonium propane
DPG-PEG: 1,2-dipalmitoyl-rac-glycero-3-methylpolyoxyethylene
DPPE-PEG: N-(methylpolyoxyethylene oxycarbonyl)-1,2-dipalmitoyl-sn-glycero-3-phosphoethanolamine
DSG-PEG: 1,2-distearoyl-rac-glycero-3-methylpolyoxyethylene
DSPC: 1,2-diastearoyl-sn-glycero-3-phosphocholine
DSPE-PEG: 1,2-distearoyl-sn-glycero-3-phosphoethanolamine-N-[methoxy(polyethylene glycol)-2000]
DSPE-PEG-Mal: 1,2-distearoyl-sn-glycero-3-phosphoethanolamine-N-[maleimide(polyethylene glycol)]
EDOPC: 1,2-dioleoyl-sn-glycero-3-ethylphosphocholine
LNP: lipid or polymer nanoparticle
LPS: lipopolysaccharides
PAHSA: (palmitoyloxy) octadecanoic acid
PAHSA-d9: [((13,13,14,14,15,15,16,16,16-d9)palmitoyl)hydroxyl]-stearic acid
polyIC: Polyinosinic:polycytidylic acid
MPLA: monophosphoryl lipid A
MVL5: N1-[2-((1S)-1-[(3-aminopropyl)amino]-4-[di(3-amino-propyl)amino]butylcarboxamido)ethyl]-3,4-di[oleyloxy]-benzamide

Antigens are toxins and similar foreign substances that may induce an immune response in the body leading to the production of antibodies and the generation of antigen-specific CTLs. Adjuvants generally act to augment a body's immune response and enhance the effectiveness of medical treatments.

According to embodiments of the present invention, vaccination compositions incorporating a nanoparticle assembly loaded with antigens, adjuvants, or both, mimic viral pathogens and have the ability to bring about multivalent presentation of antigens to maximize immune responses.

According to embodiments of the present invention, nanoparticle loading vaccination compositions promote cross-presentation by following the endocytic pathway through endosome disruption to release loaded antigens, adjuvants, or both into the cytosol, where loaded antigens can be processed and presented intracellularly to trigger CTL killing effects.

According to embodiments of the present invention, the nanoparticle is primarily lipid-based. In some embodiments, the nanoparticle is primarily polymer-based. In some embodiments, the nanoparticle may be an inorganic nanoparticle. In some embodiments, the nanoparticle may be a hybrid nanoparticle, comprised of at least two of lipid, polymer or inorganic materials.

According to embodiments of the present invention, the synthesized nanoparticle is constructed to deliver a variety of antigens and adjuvants. In some embodiments, the nanoparticle co-delivers antigens and adjuvants. In some embodiments, the nanoparticle only delivers antigens. In some embodiments, the nanoparticle only delivers adjuvants.

According to embodiments of the present invention, the antigens may be protein antigens, antigenic peptide epitopes, or antigen encoded nucleic acids. In some embodiments, the antigens are selected from a group consisting of viral, bacterial, parasitic, allergen, toxoid, tumor-specific, and tumor-associated antigens. In a preferred embodiment, the antigen is an RNA-encoding antigen.

According to embodiments of the present invention, the adjuvant incorporated by the nanoparticle is nucleic acid. In some embodiments, the adjuvant is a lipid. In some embodiments, the adjuvant is a peptide. In some embodiments, the adjuvant is a protein. In some embodiments, the adjuvant is an antibody.

According to embodiments of the present invention, there are many spatial configurations of the antigens and adjuvants to be encapsulated within or on the surface of the nanoparticle. In some embodiments, both adjuvants and antigens are encapsulated within the nanoparticle. In some embodiments, antigens are attached to the surface of the nanoparticle while adjuvants are encapsulated within the nanoparticle. In some embodiments, the antigens are attached to the surface of the nanoparticle, while the adjuvants are embedded in the membrane layer of the nanoparticle. In some embodiments, the antigens are encapsulated within the nanoparticle while the adjuvant is embedded in the membrane layer of the nanoparticle. In some embodiments, the antigens are encapsulated within the nanoparticle while the adjuvants are located on the surface of the nanoparticle. In some embodiments, the antigens are attached to the surface of the nanoparticle while the adjuvants are located on the surface of the nanoparticle. In some embodiments, antigens are embedded in the membrane layer of the nanoparticle while adjuvants are encapsulated within the nanoparticle. In some embodiments, antigens are embedded in the membrane layer of the nanoparticle while the adjuvants are located on the surface of the nanoparticle. In some embodiments, both antigen and adjuvant are embedded in the membrane layer of the nanoparticle.

According to embodiments of the present invention, peptide antigens conjugate to the surface of the nanoparticle. In some embodiments, protein antigens conjugate to the surface of the nanoparticle. In some embodiments, protein antigens are encapsulated within the nanoparticle. In some embodiments, peptide antigens are encapsulated within the nanoparticle. In some embodiments, protein antigens are embedded in the membrane layer of the nanoparticle. In some embodiments, peptide antigens are embedded in the membrane layer of the nanoparticle.

According to embodiments of the present invention, nucleic acid antigens are encapsulated within the nanoparticle. In some embodiments, nucleic acid antigens are conjugated to the surface of the nanoparticle. In some embodiments, nucleic acid antigens are embedded in the outer membrane layer of the nanoparticle.

According to embodiments of the present invention, nucleic acid adjuvants are encapsulated within the nanoparticle. In some embodiments, nucleic acid adjuvants are conjugated to the surface of the nanoparticle. In some embodiments, nucleic acid adjuvants are embedded in the outer membrane layer of the nanoparticle.

According to embodiments of the present invention, lipid adjuvants are conjugated to the surface of the nanoparticle or are embedded in the membrane layer of the nanoparticle.

According to embodiments of the present invention, peptide adjuvants or biomaterial-conjugated peptide adjuvants are encapsulated within the nanoparticle. In some embodiments, the peptide adjuvants or biomaterial-conjugated peptide adjuvants are conjugated to the surface of the nanoparticle. In some embodiments, peptide adjuvants or biomaterial-conjugated peptide adjuvants are embedded in the membrane layer of the nanoparticle.

According to embodiments of the present invention, protein adjuvants are encapsulated within the nanoparticle. In some embodiments, protein adjuvants are conjugated to the surface of the nanoparticle. In some embodiments, protein adjuvants are embedded in the membrane layer of the nanoparticle.

According to embodiments of the present invention, antibody adjuvants are encapsulated within the nanoparticle. In some embodiments, the antibody adjuvants are conjugated to the surface of the nanoparticle.

According to embodiments of the present invention, the vaccine nanoparticles may load multiple antigens and adjuvants all together in one single nanoparticle or separate nanoparticles.

### Exemplary Formation of Amph-Ag

According to embodiments of the present invention, amphiphilic lipid or polymer conjugated antigens (amph-Ag) are synthesized by reacting N-terminal cysteine-modified peptides with a lipid or polymer formulation in dimethylformamide (DMF) solvent. The reaction and resulting amph-Ag compound is evaluated and quantified using high-performance liquid chromatography (HPLC). Once the reaction is determined to be complete, the DMF solvent is dialyzed out using distilled water. The remaining compound is lyophilized to obtain solid amph-Ag. To prepare the concentrated stock solution, the amph-Ag can be dissolved in DMSO or sterilized water, and stored at −80° C. The amph-Ag will be diluted to the working concentration with sterilized water before injecting into animal subjects.

The lipid or polymer formulations that may be used to create amph-Ag include but are not limited to the maleimide functionalized DSPE-PEG, DMG-PEG, DPPE-PEG, DMPE-PEG, DSG-PEG, DPG-PEG, DOG-PEG, vitamin E (such as tocopherals and tocotrienols), and vitamin A metabolites (such as retinoic acid and all-trans retinoic acid).

### Exemplary Formation of LNP-Adjuvant

According to embodiments of the present invention, a nanoparticle incorporating one or more adjuvants (LNP-Adjuvant) is formulated by combining phospholipids, fusogenic lipids, cationic lipids containing one or more primary, and secondary or tertiary amine groups and lipid or polymer formulations such as DSPE-PEG.

In a non-limiting example, the creation of LNP-Adjuvant is facilitated by a reaction having a molar ratio of 10:48:40:2 of DSPC to cholesterol to DOTAP to DSPE-PEG. An 8:1 molar ratio of nitrogen on DOTAP to phosphate on RNA is used for the formulations. In a preferred embodiment, the reaction is facilitated by a modified ethanol dilution process. Absolute ethanol containing the lipid cocktail mentioned above is quickly mixed with equal volumes of RNA in 100 mM citrate buffer (pH of 6) by repeatedly pipetting up and down approximately twenty times, followed by immediately adding another equal volume of 100 mM citrate buffer (pH of 6) to the above mixture and repeatedly pipetting up and down approximately 80 times. This mixing step can be performed using a syringe pump to achieve spontaneous mixing. The above mixture is incubated for approximately fifteen to sixty minutes at room temperature for emulsification and equilibrium, with continuous shaking to facilitate even mixing. The entire mixture is then dialyzed in sterile phosphate buffered saline (PBS) to obtain the final LNP-Adjuvant particle.

According to embodiments of the present invention, the cationic lipids containing one or more primary, secondary, or tertiary amine groups used to create LNP-Adjuvant include but are not limited to: DOTAP, DDAB, DOTMA, MVL5, EDOPC, D-erythro-2-N-[6′-(1″-pyridinium)-hexanoyl]-sphingosine, 1,2-dipalmitoyl-sn-glycero-3-[phosphor-rac-(3-lysyl(1-glycerol))], 1,2-dioleoyl-sn-glycero-3-[phosphor-rac-(3-lysyl(1-glycerol))], and similar cationic lipids.

In some embodiments, the fusogenic lipids used to formulate the LNP are neutral lipids that promote the formation of lipid structures in order to favor the cellular uptake and promote the organization of lipids into stable bilayers able to form nanoparticles. Fusogenic lipids may also contribute to the flexibility, stability and elasticity of the monolayer membranes on lipid vesicles. Some examples of such fusogenic lipids include but are not limited to: DOPE, DOPC, 1,2-bis (10,12-tricosadiynoyl)-sn-glycero-3-phosphocholine (DC(8,9)PC), 1-palmitoyl-2-(9′-oxo-nonanoyl)-sn-glycero-3-phosphocholine, DGTS, DGTS-d9, PAHSA, PAHSA-d9, urea-ceramide, 1-hexadecyl-2-(9Z-octadecenoyl)-sn-glycero-3-phosphoethanolamine, 1-myristoyl-2-(4 nitrophenylsuccinyl)-sn-glycero-3-phosphocholine, 1-O-hexadecyl-2-oleoyl-sn-glycero-3-phosphocholine, 1-O-hexadecyl-2-(eicosatrienoyl)-sn-glycero-3-phosphocholine, 1-oleoyl-N-heptadecanoyl-D-erythro-sphingosine, N-lauroyl-1-deoxysphingosine, N-palmitoyl-1-deoxysphingosine, N-palmitoyl-1-desoxymethylsphingosine, N-lauroyl-1-desoxymethylsphingosine, N-nervonoyl-1-desoxymethylsphinganine, N-nervonoyl-1-deoxysphingosine, N-(1-adamantaneacetyl)glucosylceramide, 1-desoxymethylsphingosine, 1-deoxysphingosine, (R)-3-(3-tetradecylureido)-4-(trimethylammonio) butanoate.

According to embodiments of the present invention, a selection of specific adjuvants are able to be incorporated into and housed within the nanoparticle to create LNP-adjuvant. Some such adjuvants include but are not limited to: pathogen-associated molecular patterns (PAMPs) such as polyIC (a TLR-3 agonist), LPS, CDN (a STING (stimulator of interferon genes) agonist), lipid adjuvants such as glucopyranosyl lipid adjuvant, MPLA, Lipid A Detoxified (Salmonella minnesota R595), D-(+)-trehalose 6,6′-dibehenate, CpG (a TLR-9 agonist), similar nucleic acid adjuvant variants, and ligands having the ability to stimulate innate signaling pathways or targeting antigen-presenting cells, such as CD40 antibody. Specifically, RNA replicons may serve as adjuvants to stimulate innate immunity during the vaccination process. In some embodiments, these tested adjuvants can be separately encapsulated or co-encapsulated in the nanoparticle. In some embodiments, multiple adjuvants are co-loaded in a single nanoparticle to synergize the effects of innate immune stimulation.

It is believed that LNP-Adjuvants operate through the activation of antigen-presenting cells (APCs) in draining lymph nodes (dLNs) after immunization.

### Exemplary Formation of LNP-(Adjuvant+Antigen)

LNP-(Adjuvant+Antigen) is formulated by combining phospholipids, fusogenic lipids, cationic lipids containing one or more primary, and secondary or tertiary amine groups and the aforementioned amph-Ag.

The disclosed LNP particle may be loaded with both antigen and adjuvant components utilizing a preparation similar to the above-described preparation procedure of LNP-Adjuvant, wherein the DSPE-PEG is replaced with a combination of DSPE-PEG and amph-Ag. The ratio of the substituted DSPE-PEG and amph-Ag depends on the antigen dose in the vaccination regimen.

Specifically, according to an exemplary embodiment of the present invention, the creation of LNP-(Adjuvant+Antigen) is facilitated by a reaction having a molar ratio of 10:48:40:2 of DSPC to cholesterol to DOTAP to [DSPE-PEG and amph-Ag]. An 8:1 molar ratio of nitrogen on DOTAP to phosphate on RNA is used for the formulations. In this embodiment, the DSPE-PEG portion of the amph-Ag is inserted in the lipid layer of LNP, and the antigen portion of DSPE-PEG-antigen stretches out and tethers on the LNP particle surface.

According to embodiments of the present invention, various immune-modulatory molecules or cell targeting molecules can be incorporated in the inner layer of the nanoparticle or modified on the nanoparticle surface, such as immunomodulatory antibodies, cell targeting ligands and similar immune-modulatory molecules such as cytokines and chemokines.

### Exemplary Vaccine Preparation

According to embodiments of the present invention, a vaccine is prepared using the above-mentioned nanoparticles incorporating select antigens and adjuvants. Priming and booster immunizations are conducted by subcutaneous injection at the tail base of the animal subject. The effective vaccines contain antigens and adjuvants whose components have been described above. In some embodiments, during prophylactic vaccination, the subject is primed on day zero, boosted on day fourteen, with the subject's immune response analyzed on day twenty. On day thirty-four, 0.1 M B16F10 (melanoma tumor cells) are administered to the vaccinated subject to monitor the tumorigenesis and tumor growth progression. In some embodiments, the antigen dosage is 10-20 ug per immunization, while the adjuvant dosage is around 10 ug per immunization. The vaccines may be delivered by bilateral tail base subcutaneous injections. In some embodiments, the dosing schedule depends on the subject's cancer stage.

According to embodiments of the present invention, during therapeutic application, a subject is vaccinated on day eight and day fifteen after tumor cell inoculation, and the tumor growth progression is monitored. In some embodiments, the vaccine is administered subcutaneously. In some embodiments, multiple priming immunizations are performed during the first three weeks, and two boosting immunizations are performed during weeks twelve and twenty, respectively. In some embodiments, the immunizations are also conducted by intratumoral, intravenous or their combination administrations. One of ordinary skill in the art would appreciate that there are numerous manners in which the immunizations could be configured and embodiments of the present disclosure are contemplated for use with any configuration.

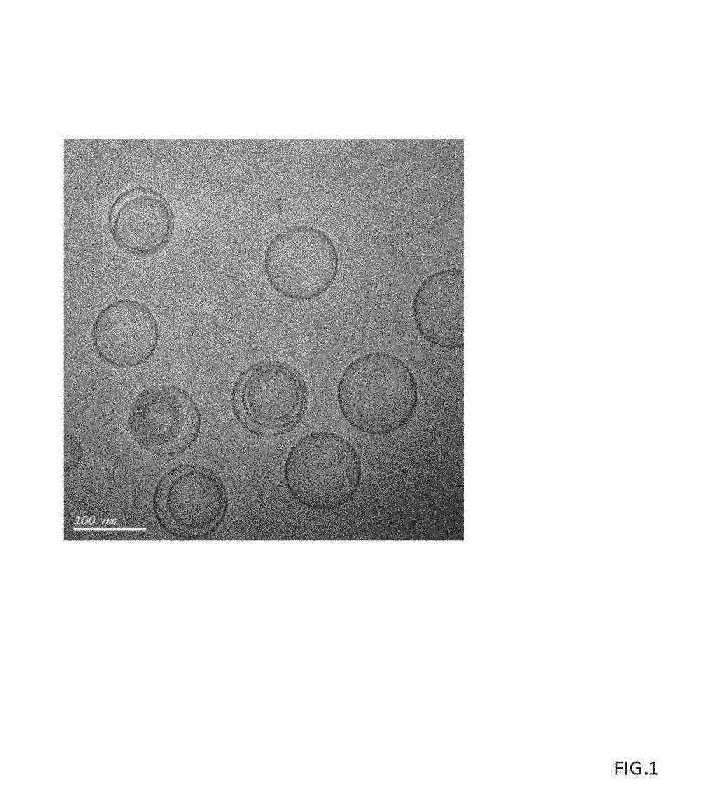

Turning now to FIGS. 1-7, a multifunctional nanoparticle in accordance with the present invention is shown.

FIG. 1 shows a Transmission Electron Microscopy (TEM) image of LNP loaded with nucleic acid adjuvants within the inner core of the LNP, where the scale bar is 100 nm.

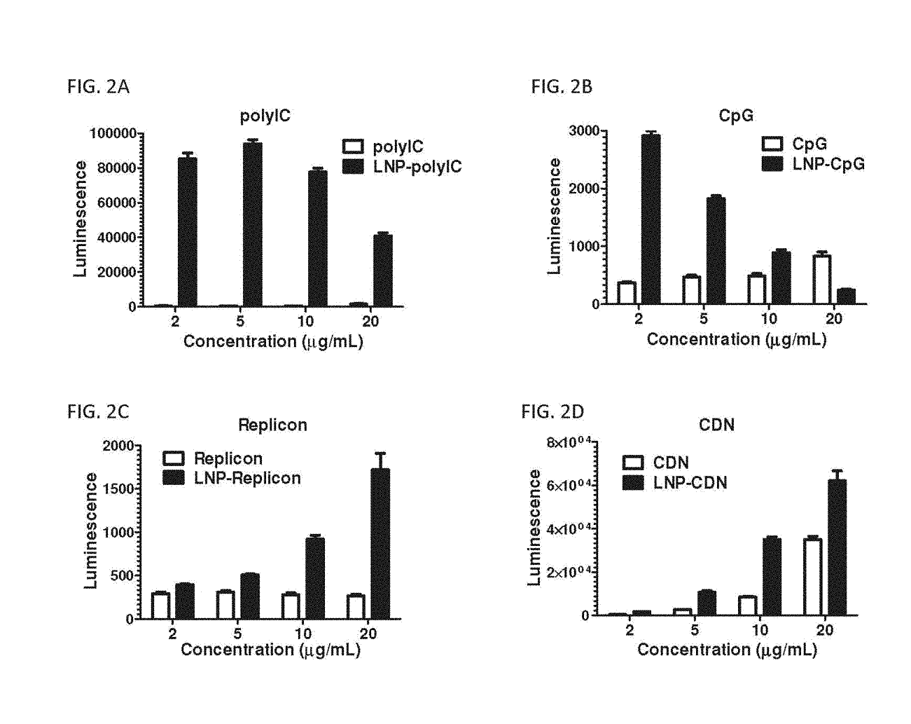

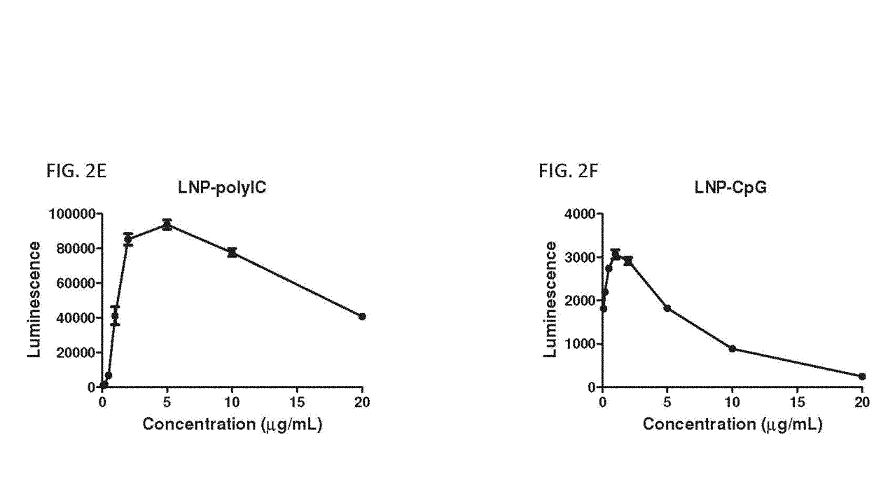

FIG. 2 is a display of graphs comparing four different nucleic acid based adjuvants (unformulated adjuvants) to LNP-Adjuvants (LNP-polyIC, LNP-CDN, LNP-CpG, and LNP-Replicon) triggering type I interferon (“IFN”) production after an incubation period of twenty-four hours in vitro. Type I IFNs are a large subgroup of interferon proteins that help regulate the activity of the immune system. RAW-Lucia ISG cell lines are used to monitor IFN activation by determining the luminescence signal from the luciferase reaction in the cell supernatant. The emitted luminescence signal measured is in proportion to the production of IFN stimulated by the tested nucleic acid based adjuvants.

As shown in FIG. 2 (A-D), the LNP-Adjuvants (LNP-polyIC, LNP-CDN, LNP-CpG, and LNP-Replicon) induced significantly higher type I IFN production than unformulated adjuvants. PolyIC is double-stranded RNA (dsRNA) and a ligand for toll-like receptor 3 (TLR-3). CpG is unmethylated single stranded DNA and a ligand for TLR-9. Replicon RNA has natural adjuvanticity due to its viral components in the construct. Cyclic dinucleotide (CDN) is a STING agonist and a potent inducer of the innate immune response. The tested concentrations ranged from 2-20 ug/ml. LNP-replicon and LNP-CDN showed a dose-dependent response, while LNP-polyIC and LNP-CpG showed decreased responses at higher dose.

As shown in FIG. 2 (E-F), the optimal dosages tested and found to induce an innate immune response are 5 ug/ml for LNP-polyIC and 1 ug/ml for LNP-CpG. It is likely the tested immune responses faded at higher doses due to the tolerogenic responses in phagocytes.

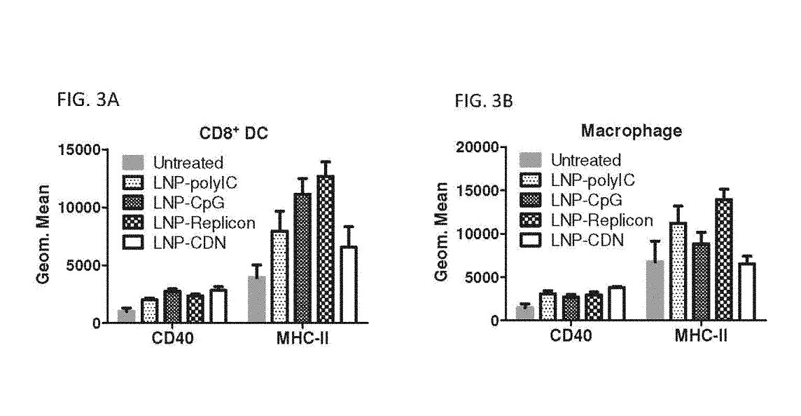

Referring now to FIG. 3 (A-B), two graphs are shown demonstrating that LNP-Adjuvants stimulate the activation of antigen-presenting cells (APCs) in draining lymph nodes (dLNs) after immunization. The LNP-Adjuvants used to perform this test were LNP-polyIC, LNP-CpG, LNP-Replicon, and LNP-CDN. The data presented in FIG. 3 was collected after C57BL/6 mice were immunized subcutaneously, at the tail base, with DSPE-PEG-conjugated melanoma peptide antigen (amph-Ag) containing 10 ug peptide antigens, and LNP-Adjuvants, containing 10 ug adjuvants. One day later, dLNs were dissected and enzymatically digested to single cell suspensions using 0.8 mg/ml collagenase/Dispase and 0.1 mg/ml DNase in RPMI-1640 serum-free media. The expression levels of activation markers (CD40, MHC-II) on APCs (cross-presenting dendritic cell (DC), macrophage) were analyzed by flow cytometry. The data collected shows that the tested LNP-Adjuvants promote the activation of APCs in dLNs. The lymph nodes (LN) evaluated for the test demonstrated by FIG. 3 underwent a digestion process, whereby: (1) The LN was pierced once with forceps to break the LN capsule; (2) the digestion media was prepared using: 0.8 mg/ml collagenase/Dispase abd 0.1 mg/ml DNase in RPMI-1640 serum-free media; (3) the LN was transferred into an Eppendorf tube and 0.5 ml of freshly made digestion media is added to the mixture (if more LNs are processed in the same tube, the volume of the digestion media may be increased in the same proportion); (4) the Eppendorf tube was incubated in 37 degree Celsius water bath and gently inverted at five minute intervals to ensure that the contents were well mixed; (5) after fifteen minutes, the LNs were aspirated and expirated multiple times using a 16 G needle until no additional cells were released. Each LN was forced through the pipette to disrupt the LN capsule and release leukocytes; (6) the large fragments are allowed to settle for thirty seconds and the supernatant is transferred to 10 ml ice-cold FACS buffer (a second round of digestion may be performed in a similar manner to produce additional supernatant); and (7) the cell pellet was washed with FACS buffer, ready to be stained with antibodies.

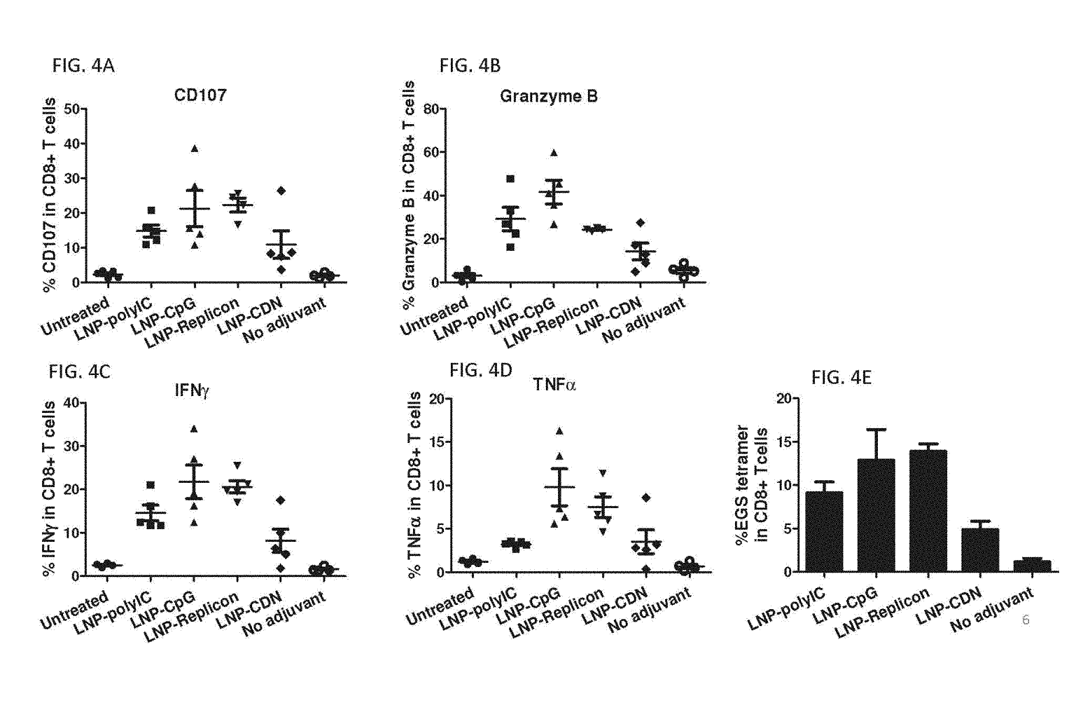

Referring now to FIG. 4(A-E), five graphs are shown demonstrating that immunizations consisting of amph-Ag, having a peptide antigen sequence: AVGALEGPRNQDWLGVPRQL (hereinafter referred to as amph-EGP), and the LNP-Adjuvants promote antigen-specific T-cell response. The LNP-Adjuvants used for this test were LNP-polyIC, LNP-CpG, LNP-Replicon, and LNP-CDN. The data in FIG. 4 was collected after C57BL/6 mice were immunized with amph-EGP. The C57BL/6 mice were immunized with LNP-Adjuvants (10 ug adjuvant per immunization) and amph-EGP (10 ug peptide antigen per immunization) subcutaneously at the tail base on day 0 and 14. Bilateral immunization was used at the tail base to increase the number of draining lymph nodes (dLNs) and possibly engage twice as many antigen-specific immune cells. On day 20, peripheral blood was collected and processed to peripheral blood mononuclear cells (PBMCs). The PBMCs were stimulated ex vivo with cognate EGS peptide (2 ug/ml) of the sequence EGSRNQDWL (hereinafter referred to as EGS) for six hours, with the presence of brefeldin A (5 ug/ml) in the last four hours to block protein secretion before the flow cytometry analysis.

As demonstrated by the data recorded in FIGS. 4(A-E), the tested LNP-Adjuvants increased the expressions of degranulation marker CD107 in CD8⁺ T-cells, as shown in FIG. 4A, and cytolytic enzyme granzyme B in CD8⁺ T-cells, as shown in FIG. 4B, compared to the simultaneously tested amph-EGP and the untreated groups, suggesting that use of the LNP-Adjuvants enhance the effective killing of tumor cells.

As shown in FIGS. 4C-4D, after ex vivo stimulation, the tested LNP-Adjuvants induced significant production of pro-inflammatory cytokines IFNγ (FIG. 4C) and TNFα (FIG. 4D) in CD8⁺ T-cells than amph-EGP and untreated groups, suggesting enhanced antigen-specific T-cell effector function. Referring now to FIG. 4E, immunizations with LNP-Adjuvants triggered more tetramer positive CD8⁺ T-cells in peripheral blood than amph-EGP and untreated groups, suggesting significant clonal expansion of antigen-specific CD8⁺ T cell.

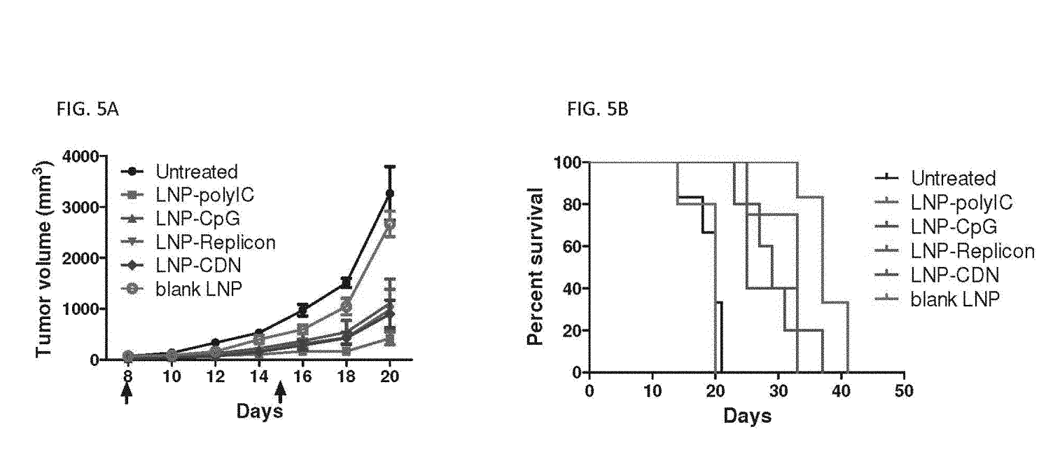

Referring now to FIG. 5, two graphs demonstrating that the disclosed LNP-adjuvants inhibit tumor progression in melanoma-bearing mice when immunizing with amph-EGP are shown. C57BL/6 mice were subcutaneously inoculated with 0.5 million B16F10 cells in the right flank on day 0. The melanoma-bearing C57BL/6 mice were immunized with amph-EGP, together with blank LNP as a control and four different LNP-Adjuvants (LNP-polyIC, LNP-CpG, LNP-Replicon and LNP-CDN), each immunization bearing 10 ug adjuvant and 10 ug peptide antigen, subcutaneously at the tail base on day 8 and 15 post tumor inoculation. Bilateral immunization was used at the tail base. FIG. 5A is a graph showing the tumor volumes measured every day with a caliper. The tumor volumes were calculated using the formula: V (mm³)=½ width\hat{ }2×length. The survival rate of the melanoma-bearing mice was monitored. FIG. 5B is a graph showing the survival rate of the melanoma-bearing mice after the immunizations, demonstrating that the LNP-Adjuvants retarded tumor progression and prolonged survival.

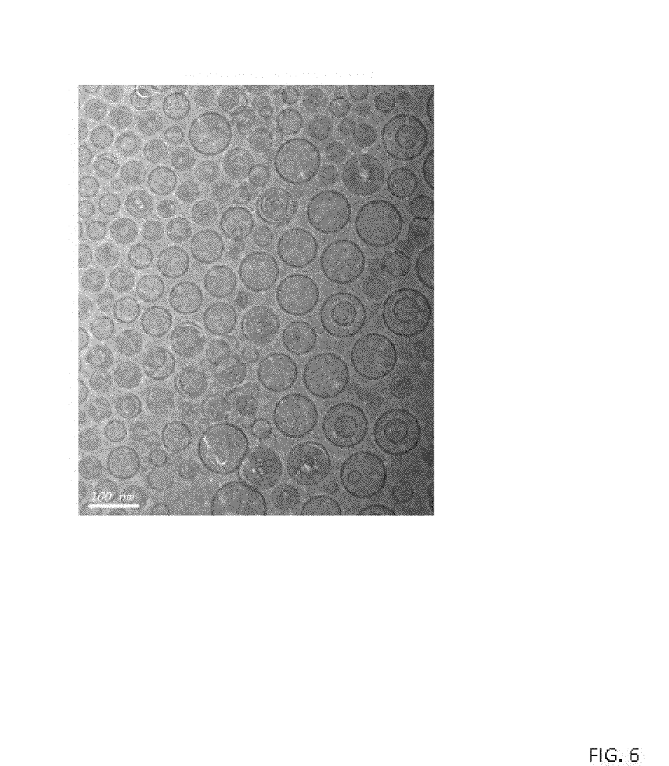

FIG. 6 shows a Transmission Electron Microscopy (TEM) image of LNP-(polyIC+(amph-Ag)) wherein the antigen and adjuvant loading nanoparticle contains nucleic acid adjuvants within its inner core, and peptide antigens tethered to its surface, where the scale bar is 100 nm.

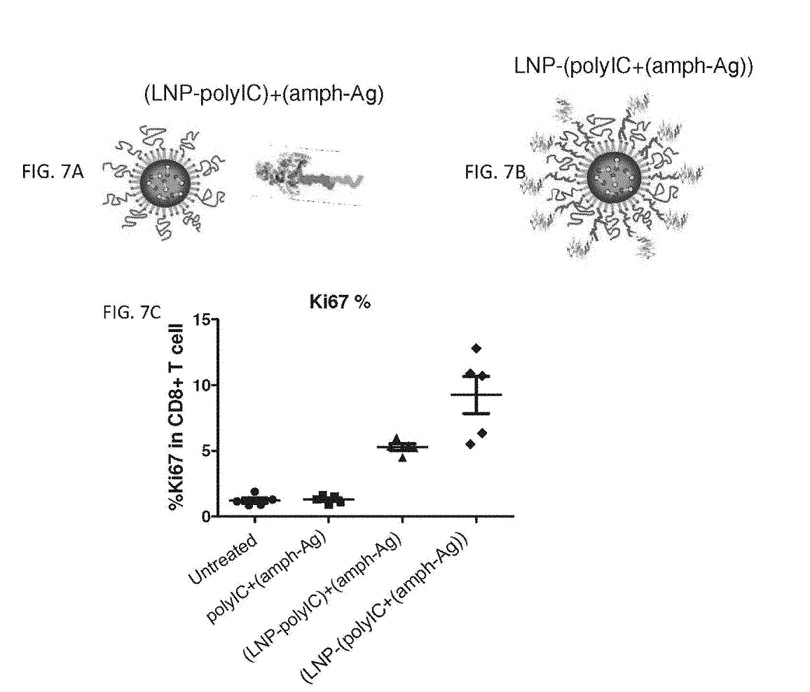

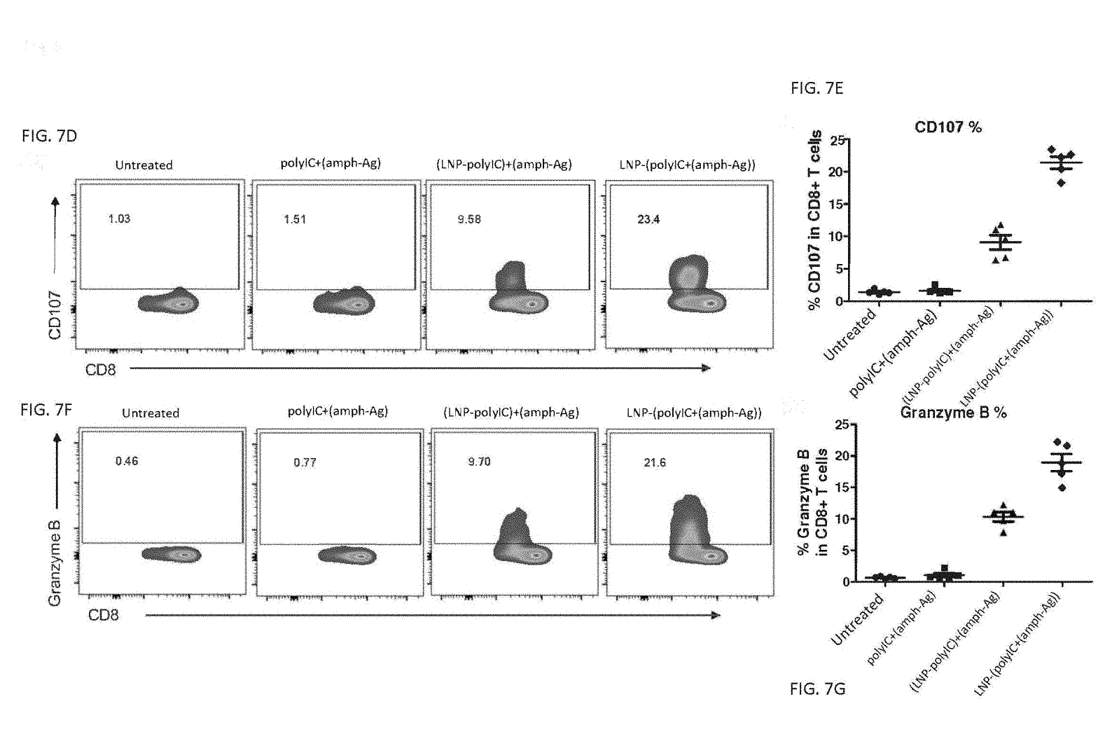

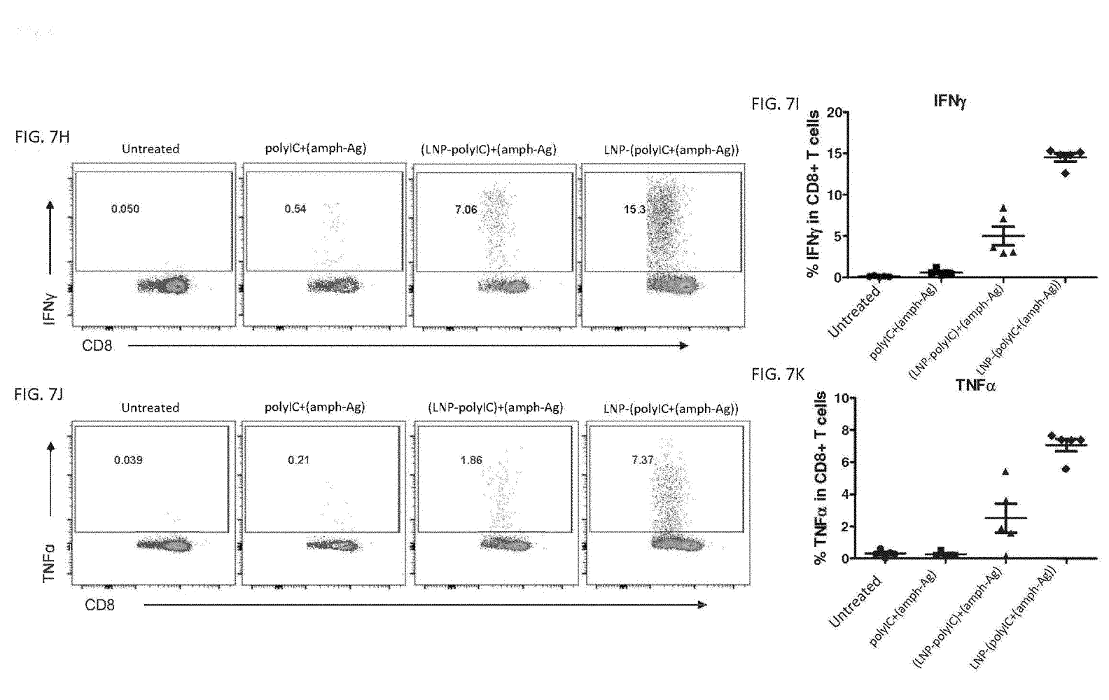

FIG. 7A is an illustration of separate antigen (Ag) and adjuvant components ((LNP-polyIC)+(amph-Ag)). FIG. 7B is an illustration of an antigen (Ag) and adjuvant combined nanoparticle (LNP-(polyIC+(amph-Ag)).

FIG. 7(C-K) are graphs demonstrating that immunizations with antigen and adjuvant combined nanoparticles (LNP-(polyIC+(amph-Ag)), where the adjuvant is loaded inside the LNP, with the antigen tethered on the surface of the same LNP, displayed stronger antigen-specific immune responses than immunization with separate antigen and adjuvant components ((LNP-polyIC)+(amph-Ag)), where the adjuvant is loaded inside the LNP, and the antigen is conjugated in a separate amphiphilic lipid or polymer chain. Unformulated adjuvants were tested as a control and elicited little immune responses after immunization with amph-EGP. The data was collected after C57BL/6 mice were immunized with (1) unformulated polyIC and amph-EGP (polyIC+(amph-Ag)), (2) (LNP-polyIC)+(amph-Ag), and (3) LNP-(polyIC+(amph-Ag)) subcutaneously at the tail base on day 0 and 14. Bilateral immunization were used at the tail base. The vaccine doses for all immunizations were 10 ug polyIC and 10 ug EGP peptide. On day 20, peripheral blood was collected and processed to peripheral blood mononuclear cells (PBMCs). PBMCs were stimulated ex vivo with 2 ug/ml EGS, for six hours, with 5 ug/ml brefeldin A added during the last four hours to block any protein secretion before the flow cytometry could be analyzed. LNP-(polyIC+(amph-Ag)) elicited the strongest antigen-specific immune responses, compared to polyIC+(amph-Ag) and (LNP-polyIC)+(amph-Ag) groups. This conclusion is evidenced by the data collected and demonstrated in FIG. 7(C-K). FIG. 7C is a graph of the expression of proliferation marker Ki67 in CD8⁺ T-cells, which suggests an enhanced CD8⁺ T cell proliferation upon activation. FIG. 7D and FIG. 7E are graphs of the expression of the T-cell degranulation marker CD107 in CD8⁺ cells. FIG. 7F and FIG. 7G are graphs of the expression of cytolytic enzyme granzyme B in CD8⁺ cells, suggesting enhanced CTL cytolytic activity and tumor cell killing effect. FIGS. 7H and I are graphs of the production of pro-inflammatory cytokines IFNγ in CD8⁺ cells. FIG. 7J and FIG. 7K are graphs of the production of pro-inflammatory cytokines TNFα in CD8⁺ T cells. FIGS. 7(H-K) suggest enhanced antigen-specific T-cell effector function. The data collected during and after the aforementioned examinations show that the immune responses in the LNP-(polyIC+(amph-Ag)) group were almost one-fold higher than those of (LNP-polyIC)+(amph-Ag) group, and polyIC+(amph-Ag) group showed little immune responses after both prime and boost immunizations.

1. A vaccine composition for inducing an immune response comprising of a nanoparticle, at least one antigen and at least one adjuvant.

2. The vaccine composition of claim 1, wherein said nanoparticle is a hybrid comprising of any combination of the following: a lipid-based nanoparticle, a polymer-based nanoparticle, a biomaterial conjugate, and an inorganic-based nanoparticle.

3. The vaccine composition of claim 1, wherein said antigen is a peptide or a protein.

4. The vaccine composition of claim 1, wherein said antigen is a nucleic acid or an RNA-encoding antigen.

5. The vaccine composition of claim 1, wherein said antigen is a nucleic acid encoding viral, bacterial, parasitic, allergen, toxoid, tumor-specific, tumor associated antigens, or neoantigens.

6. The vaccine composition of claim 1, wherein said antigen is encapsulated within an inner core of said nanoparticle.

7. The vaccine composition of claim 1, wherein said antigen is embedded in the lipid bilayer of said nanoparticle.

8. The vaccine composition of claim 1, wherein said antigen is conjugated to the surface of said nanoparticle.

9. The method of claim 1, wherein said adjuvant is a nucleic acid.

10. The vaccine composition of claim 1, wherein said adjuvant is a derivative or combination nucleic acid.

11. The vaccine composition of claim 1, wherein said adjuvant is a RNA replicon derived from either positive- or negative-strand RNA viruses.

12. The vaccine composition of claim 1, wherein said adjuvant is a peptide or a protein.

13. The vaccine composition of claim 1, wherein said adjuvant is an antibody.

14. The vaccine composition of claim 1, wherein said adjuvant is a lipid.

15. The vaccine composition of claim 1, wherein said adjuvant is encapsulated within an inner core of said nanoparticle.

16. The vaccine composition of claim 1, wherein said adjuvant is embedded in the lipid bilayer of said nanoparticle.

17. The vaccine composition of claim 1, wherein said adjuvant is conjugated to the surface of said nanoparticle.

18. A method of inducing an immune response to an antigen comprising: administering, to a subject, a vaccine composition comprising a nanoparticle, at least one adjuvant and at least one antigen.

19. The method of inducing an immune response in accordance with claim 18, wherein said nanoparticle induces an innate immune response by stimulating the activation of antigen-presenting cells in draining lymph nodes.

20. The method of inducing an immune response in accordance with claim 18, wherein said nanoparticle induces adaptive immune responses directed against target antigens, by enhancing the production of tetramer positive CD8⁺ T cells.

21. The method of inducing an immune response in accordance with claim 18, wherein said nanoparticle induces adaptive immune responses directed against target antigens, by enhancing the production of degranulation marker CD107 and cytolytic enzyme granzyme B in CD8⁺ T cells.

22. The method of inducing an immune response in accordance with claim 18, wherein said nanoparticle induces adaptive immune responses directed against target antigens, by enhancing the production of pro-inflammatory cytokines in CD8⁺ T cells.

23. A method of inducing an immune response in a subject comprising: administering, to a subject, a vaccine composition comprising at least one adjuvant encapsulated within an inner core of said nanoparticle, with at least one antigen tethered to a surface of said nanoparticle.

24. A method of inducing an immune response in a subject comprising: administering, to a subject, a vaccine composition comprising at least one antigen loaded inside a nanoparticle.
